(19) United States
(12) Patent Application Publication
(10) Pub. No.: US 2007/0287159 A1
(43) Pub. Date: Dec. 13, 2007

(54) Miniature lid for saving antibody

(76) Inventor: Stephen Liye Chen, El Monte, CA (US)

Correspondence Address: Stephen Chen, 12331 Felipe Street, El Monte, CA, 91732, US

(21) Appl. No.: 11/451,024

(22) Filed: Jun. 12, 2006

(51) Int. Cl. G01N 33/53; C12M 3/00

(52) U.S. Cl. 435/7.1; 435/287.2

(57) ABSTRACT

A miniature lid is introduced to dramatically reduce antibody consumption. The miniature lid covers a specimen and force antibody solution to form a 1.5 mm thin layer so that specimen can be immersed completely in 0.1 ml volume.

### FIELD OF THE INVENTION

The present invention relates generally to devices for antibody immune detection in biotechnology. More specifically, it is a device for reducing antibody consumption.

### BACKGROUND OF THE INVENTION

Biologic molecules, such as DNA and proteins, are invisible by human eyes. Their visualization in specimen via liquid reagent is one of the most important procedures in modern biotechnology. For example, a specific antibody can be used to detect a target protein in a tissue. A labeled DNA probe can be used to detect a specific DNA sequence in a Southern blot. For best detection, antibodies should be used in liquid solution at a certain concentration. A large volume of antibody solution is usually required to immerse specimen completely in a liquid container for the detection.

Antibody is the most expensive reagent in immune detection process due to difficulties of their production. Attempt has been made to reduce consumption of antibodies. Electron Microscopy Sciences Company, from its website, sells a pap pen for drawing a hydrophobic mark around specimen on slides. Antibody solution forms a large drop of liquid within the area of the mark. Unfortunately, pap pen introduces risks of uncertainty and failure:
1. It exposes specimen to dry air for making the mark, which is extremely risky and causes high background signal.
2. It fails to secure the large drop of liquid reagent in position. Moving and shaking can spill the antibody.
3. It fails to prevent evaporation of the liquid reagent, which changes antibody concentration and even exposes specimen to dry air during antibody incubation.
4. A special chemical solvent is required to remove the hydrophobic mark.

Heavy consumption of antibody is a long-felt problem but remains unsolved.

### SUMMARY OF THE PRESENT INVENTION

It is, therefore, an object of the invention to provide an easy device for reducing antibody consumption.

The advantages of the invention over prior art can be summarized as follows:
(1) It is easy. A miniature lid can be simply placed anywhere to cover a specimen.
(2) It is reliable. The miniature lid holds antibody solution in position and blocks its evaporation.
(3) It saves antibody. 95% of antibody can be saved using the miniature lid.
(4) It makes washing step faster and easier after antibody incubation.

### BRIEF DESCRIPTION OF THE DRAWINGS

FIG. 1 is a perspective view of an embodiment of the invention.

FIGS. 2a and 2b are illustrative diagram showing the principle of the invention.

### DETAILED DESCRIPTION OF PREFERRED EMBODIMENT

Specimens are usually attached to a support surface of containers, such as dishes, wells, or slides. A full immersion of specimen in antibody solution is required for interaction between antibody and specimen. It means that the size of the container is actually the key parameter in determining the amount of antibody used.

A miniature lid is the invention to save antibody.

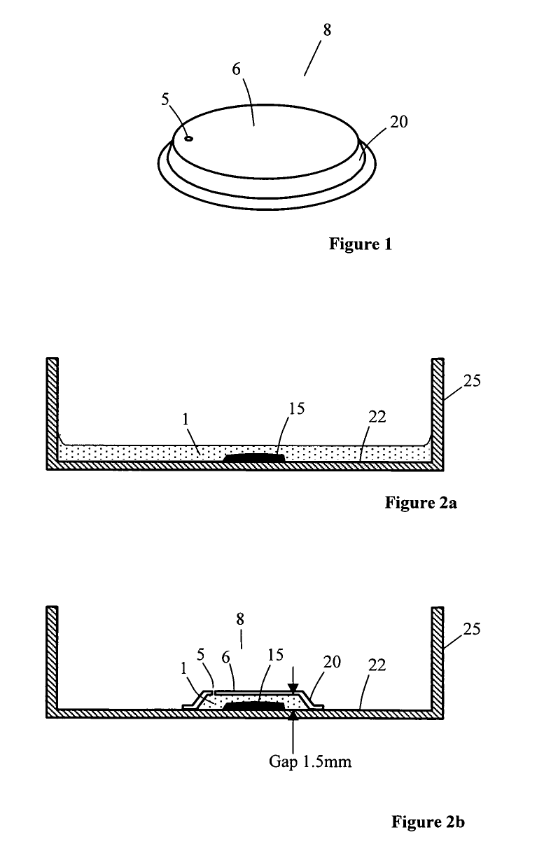

FIG. 1 shows a lid 8 for reducing antibody consumption. It is made with liquid impermeable materials, such as plastics or thin sheet metals. Flange 20 is formed around edges of sheet 6. A hole 5 is built on sheet 6.

FIGS. 2a and 2b illustrate how lid 8 works to save antibody 1. In FIG. 2a, A specimen 15 is fixed on bottom 22 of a culture dish 25. The required volume of antibody 1 is about 3 ml even though the majority area of bottom 22 is empty.

FIG. 2b is a comparison to FIG. 2a. Lid 8 is first placed on bottom 22 to cover specimen 15. Antibody 1, a liquid reagent, is then introduced via hole 5 to immerse specimen 15. Sheet 6 defines a boundary on top of specimen 15, which limits antibody 1 to distribute into a thin layer around specimen 15. By using lid 8, 0.1 ml of antibody 1 is enough to cover specimen 15. That is, the consumption of antibody 1 is reduced from 3 ml to 0.1 ml.

Flange 20 functions as a support of sheet 6. Flange 20 raises sheet 6 away from bottom 22 to form a gap in 1.5 mm. Antibody 1 contacts sheet 6 directly. The 1.5 mm gap between sheet 8 and bottom 22 is chosen for the purposes of:
1. Preventing damages to specimen 15.
2. Utilizing liquid capillary action of antibody 1 between sheet 6 and bottom 22. The force of capillary action adsorbs lid 8 against bottom 22 and holds lid 8 in position. The force of capillary action also retains the liquid of antibody 1 within the boundary of lid 8.

Liquid evaporation of antibody 1 has been a problem in prior art. The invention solves the problem by enclosing antibody 1 within lid 8 for the duration of antibody incubation. Desired certain concentration of antibody 1 can be maintained substantially.

Washing is also an important step in the procedure of antibody detection. Several changes of fresh washing solution are required after antibody incubation. In the invention, Antibody 1 is limited within the covered area around specimen. Other uncovered areas of culture dish 25 remains clean with no antibody 1. To wash specimen 15, Lid 8 will be removed after incubation of antibody 1. Then, 10 ml washing solution is added to culture dish 25 to wash specimen 15. It is a 100-fold volume increase, from 0.1 ml antibody 1 to 10 ml washing solution. As a result, the washing of specimen 15 is much cleaner and faster.

The size and shape of lid 8 should be determined by its application. Area of sheet 6 should be larger than specimen 15 in order to cover it completely. For viewing in microscopy, the shape of lid 8 can be made as a circle in 10 mm, 20 mm, or a rectangle in 22 mm×55 mm. But regardless of its sizes, the gap between sheet 6 and bottom 22 should be maintained within 1 mm-3 mm for best utilizing the force of capillary action.

Hole 5 is an optional structure for releasing air under sheet 6 or for filling antibody 1, depending on the habit of users. To minimize liquid evaporation through hole 5, the diameter of hole 5 should be 1 mm or less, which ensures 99%-99.9% of liquid surface of antibody 1 being blocked from evaporation. Multiple holes might be used when area of sheet 6 is increased.

Lid 8 has a consistent and specified volume to fill. Users can use a lab pipette to add exact amount of antibody 1 as indicated by specification.

The operation of the miniature lid can be summarized as follows:
1. Add fixing reagent to culture dish 25 to fix specimen 15. Then remove fixing reagent.
2. Add blocking reagent to culture dish 25 to block non-specific binding groups of specimen 15. Then remove blocking reagent.
3. Place lid 8 to cover specimen 15 and introduce antibody 1 in a small and specified volume into lid 8 to immerse specimen 15.
4. Remove lid 8 after incubation of antibody 1 against specimen 15.
5. Add washing solution in large volume to wash specimen 15.

Although the descriptions above contains specifications, it is apparent to those who skilled in the art that a number of other variations and modifications can be made to the invention without departing from its spirit and scope. Flange 20, for example, can be replaced by other means of structure to raise sheet 6. Hole 5 can be omitted from lid 8. Sheet 6 can be made in a variety of shapes. The gap distance between sheet 6 and bottom 22 can be smaller than 1 mm. Therefore, descriptions as set out above should not be constructed as limiting the scope of the invention but as merely providing illustration of the presently preferred embodiment of the invention.

What is claimed is:

1. A device for detection of a specimen on a support using a liquid reagent, comprising;
a sheet, made with impermeable material in a shape suitable to cover said specimen, permitting delivery of said liquid reagent to react with said specimen under said sheet, utilizing a force of capillary action to retain said liquid reagent around said specimen, blocking evaporation of said liquid reagent substantially to prevent dryness of said specimen; and
lifting means, located between said sheet and said support, raising said sheet apart from said support to form a gap for hosting said liquid reagent.

2. The device of claim 1 wherein said liquid reagent contains antibody.

3. A device for saving liquid reagent in detection of a specimen on a support, comprising;
a lid, made with impermeable material in a shape suitable to cover and to protect said specimen, permitting delivery of said liquid reagent to react with said specimen under said lid, contacting said liquid reagent directly, utilizing liquid capillary action to retain said liquid reagent into a thin layer around said specimen, blocking evaporation of said liquid reagent substantially to prevent dryness of said specimen.

4. The device of claim 3 wherein the thickness of said thin layer of said liquid reagent ranges around 1.5 mm.

5. A method for saving antibody in an immune detection of a specimen on a support, comprising steps of:
(a) treating said specimen with chemical reagents;
(b) removing said chemical reagents;
(c) having a lid, made with impermeable material in a shape suitable to cover and to protect said specimen, permitting delivery of said antibody to detect said specimen under said lid, contacting said antibody directly, utilizing liquid capillary action to retain said antibody into a thin layer around said specimen, blocking evaporation of said antibody substantially to prevent dryness of said specimen;
(d) placing said lid on said support to cover said specimen;
(e) introducing said antibody into said lid to immerse said specimen;
(f) removing said lid after incubation of said antibody for washing said specimen.
